(19) United States
(12) Patent Application Publication
(10) Pub. No.: US 2011/0160308 A1
(43) Pub. Date: Jun. 30, 2011

(54) Use of Monoamine Oxidase Inhibitors to Treat Outer Retina Disorders

(75) Inventors: Robert J. Collier, JR., Arlington, TX (US); Michael A. Kapin, Arlington, TX (US); John M. Yanni, Burleson, TX (US)

(73) Assignee: ALCON RESEARCH, LTD., Fort Worth, TX (US)

(21) Appl. No.: 12/375,247

(22) PCT Filed: Jul. 27, 2007
(86) PCT No.: PCT/US2007/074603

§ 371 Date: Jan. 27, 2009

Related U.S. Application Data
(60) Provisional application No. 60/820,735, filed on Jul. 28, 2006.

(51) Int. Cl. A61K 31/165; A61P 27/02

(52) U.S. Cl. 514/620

(57) ABSTRACT

Compositions and methods for treating disorders of the outer retina with compounds that inhibit monoamine oxidase are disclosed.

### BACKGROUND OF THE INVENTION

1. Field of Invention

The present invention is directed to compounds which are inhibitors of monoamine oxidase and their use in treating disorders of the outer retina resulting from acute or chronic degenerative conditions or diseases of the eye.

2. Description of Related Art

Age-related macular degeneration (AMD) is the leading cause of blindness in the is elderly, with an incidence of about 20% in adults 65 years of age increasing to 37% in individuals 75 years or older. Non-exudative AMD is characterized by drusen accumulation and atrophy of rod and cone photoreceptors in the outer retina, retinal pigment epithelium (RPE), Bruch's membrane and choriocapillaris; while exudative AMD leads to choroidal neovascularization (Green and Enger, Ophthalmol, 100:1519-35, 1993; Green et al., Ophthalmol, 92:615-27, 1985; Green and Key, Trans Am Ophthalmol Soc, 75:180-254, 1977; Bressler et al., Retina, 14:130-42, 1994; Schneider et al., Retina, 18:242-50, 1998; Green and Kuchle (1997). In: Yannuzzi, L. A., Flower, R. W., Slakter, J. S. (Eds.) Indocyanine green angiography. St. Louis: Mosby, p. 151-6). Retinitis pigmentosa (RP) represents a group of hereditary dystrophies characterized by rod degeneration with secondary atrophy of cone photoreceptors and underlying pigment epithelium. (Pruett, Trans Am Ophthalmol Soc, 81:693-735, 1983; Heckenlively, Trans Am Ophthalmol Soc, 85:438-470, 1987; Pagon, Sur Ophthalmol, 33:137-177, 1988; Berson, Invest Ophthalmol Vis Sci, 34:1659-1676, 1993; Nickells and Zack, Ophthalmic Genet, 17:145-65, 1996). The pathogenesis of retinal degenerative diseases, such as AMD and RP, is multifaceted and can be triggered by environmental factors in normal individuals or in those who are genetically predisposed. To date more than 100 genes have been mapped or cloned that may be associated with various outer retinal degenerations.

Light exposure is an environmental factor that has been identified as a contributing factor to the progression of retinal degenerative disorders such as AMD (Young, Sur Ophthal, 32:252-269, 1988; Taylor, et al., Arch Ophthal, 110:99-104, 1992; Cruickshank, et al., Arch Ophthal, 111:514-518, 1993). Photo-oxidative stress leading to light damage to retinal cells has been shown to be a useful model for studying retinal degenerative diseases for the following reasons: damage is primarily to the photoreceptors and retinal pigment epithelium (RPE) of the outer retina, the same cells that are affected in heredodegenerative diseases (Noell et al., Invest Ophthal Vis Sci, 5, 450-472, 1966; Bressler et al., Sur Ophthal, 32, 375-413, 1988; Curcio et al., Invest Ophthal Vis Sci, 37, 1236-1249, 1996); apoptosis is the cell death mechanism by which photoreceptor and RPE cells are lost in AMD and RP, as well as following a photo-oxidative induced cell injury (Ge-Zhi et al., Trans AM Ophthal Soc, 94, 411-430, 1996; Abler et al., Res Commun Mol Pathol Pharmacol, 92, 177-189, 1996; Nickells and Zack, Ophthalmic Genet, 17:145-65, 1996); light has been implicated as an environmental risk factor for progression of AMD and RP (Taylor et al., Arch Ophthalmol, 110, 99-104, 1992; Naash et al., Invest Ophthal Vis Sci, 37, 775-782, 1996); and therapeutic interventions which inhibit photo-oxidative injury have also been shown to be effective in animal models of heredodegenerative retinal disease (LaVail et al., Proc Nat Acad Sci, 89, 11249-11253, 1992; Fakforovich et al., Nature, 347, 83-86, 1990; Frasson et al., Nat. Med. 5, 1183-1187, 1990).

A number of different compound classes have been identified in various animal models that minimize retinal photo-oxidative injury. They include: antioxidants such as ascorbate (Organisciak et al., Invest Ophthal Vis Sci, 26:1589-1598, 1985), dimethylthiourea (Organisciak et al., Invest Ophthal Vis Sci, 33:1599-1609, 1992; Lam et al., Arch Ophthal, 108:1751-1752, 1990), α-tocopherol (Kozaki et al., Nippon Ganka Gakkai Zasshi, 98:948-954, 1994) and β-carotene (Rapp et al., Cur Eye Res, 15:219-232, 1995); calcium antagonists such as flunarizine (Li et al., Exp Eye Res, 56: 71-78, 1993; Edward et al., Arch Ophthal, 109, 554-622, 1992; Collier et al., Invest Ophthal Vis Sci, 36:S516); growth factors such as basic-fibroblast growth factor, brain derived nerve factor, ciliary neurotrophic factor, and interleukin-1-β (LaVail et al., Proc Nat Acad Sci, 89, 11249-11253, 1992); glucocorticoids such as methylprednisolone (Lam et al., Graefes Arch Clin Exp Ophthal, 231, 729-736, 1993) and dexamethasone (Fu et al., Exp Eye Res, 54, 583-594, 1992); iron chelators such as desferrioxamine (Li et al., Cur Eye Res, 2, 133-144, 1991); NMDA-antagonists such as eliprodil and MK-801 (Collier et al., Invest Ophthal Vis Sci, 40:S159, 1999).

Monoamine oxidase (MAO) inhibitors have been shown to inhibit induction of apoptosis. This inhibition is thought to result from altering gene expression for the scavenger proteins Cu/Zn superoxide dismutase (SOD1) and MN superoxide dismutase (SOD2) as well as the onco-genes Bcl-2, Bax, nitrix oxide synthase, c-JUN and nicotinamide adenine dinucleotide dehydrogenase. Rasagiline (1 mg/kg), a MAO-B inhibitor, has been shown to significantly accelerate the recovery of motor function and spatial memory in a mouse closed head injury model. Additionally, cerebral edema was reduced 40-50%. Certain other MAO inhibitors have been described for other disorders. In the retina, the MAO inhibitors selegiline and desmethylselegiline have been shown to protect ganglion cells from NMDA-induced excitotoxicity (Takahata et al., Eur J Pharmacol, 458(1-2):81-9, 2003). Deprenyl has also been shown to protect ganglion cells following optic nerve crush (Buys et al., Cur Eye Res, 14(2):119-126, 1995) or serum deprivation (Ragaiey et al., J Ocul Pharmacol Ther, 13(5):479-88, 1997). The effect of clorgyline, a MAO-A inhibitor, on photoreceptor rhythms of disk shedding and autophagic degradation has been reported (Reme et al., Trans Ophthalmol Soc U K, 103 (Pt 4):405-10, 1983.).

U.S. Pat. No. 5,263,957 describes N-phenylalkyl substituted α-amino carboxamide derivatives. The compounds described are said to be useful as antiepileptic, anti-Parkinson, neuroprotective, antidepressant, antispastic, and/or hypnotic agents. The '957 patent does not mention the use of such compounds for treating disorders of the outer retina. In fact, ophthalmic indications are not mentioned at all.

U.S. Pat. No. 5,945,454 describes 2-(4-substituted)-benzylamino-2-methyl-propanamides and their use as therapeutic agents. The compounds are described as being active on the central nervous system and are suggested for use in disorders of the central nervous system, including ocular damage or retinopathy. Significantly, the compounds of this invention are not encompassed within the compounds claimed for use in the described methods.

U.S. Pat. No. 5,242,950 describes a method for treating macular degeneration by administering L-deprenyl or a salt thereof. L-deprenyl is a selective MAO-B inhibitor. The L-deprenyl is to be administered orally or transdermally. The '950 patent does not suggest the use of other types of MAO inhibitors, nor does it suggest delivery methods other than oral or transdermal. U.S. Pat. No. 5,981,598 describes a method for treating glaucoma by administering a deprenyl compound. The compounds disclosed for use in the to methods of the '598 patent or the '950 patent differ significantly from the compounds of this invention. In fact, “deprenyl compound” is defined in the '598 patent as “deprenyl compounds which are structurally similar to deprenyl,” thus excluding the preferred compounds of the invention.

WO 2005/039591 describes benzazepine derivatives, which are MAO-B inhibitors. The compounds described therein, again, differ significantly from the compounds of this invention.

None of the above-described publications mention the use of the compounds of this invention to inhibit or prevent retinal degeneration resulting from loss or damage to photoreceptors and/or retinal pigment epithelium cells. What is needed are more effective compounds and methods of treatment for these serious, sight-threatening disorders.

### SUMMARY OF THE INVENTION

The present invention is directed to MAO-A/B and B inhibitors which have been discovered to be useful in treating disorders of the outer retina, particularly: AMD; RP and other forms of heredodegenerative retinal disease; retinal detachment and tears; macular pucker; ischemia affecting the outer retina; diabetic retinopathy; damage associated with laser therapy (grid, focal, and panretinal) including photodynamic therapy (PDT); trauma; surgical (retinal translocation, subretinal surgery, or vitrectomy) or light-induced iatrogenic retinopathy; and preservation of retinal transplants. As used herein, the outer retina includes the RPE, photoreceptors, Muller cells (to the extent that their processes extend into the outer retina), and the outer plexiform layer. The compounds are formulated for systemic or local ocular delivery.

### BRIEF DESCRIPTION OF THE DRAWINGS

The following drawings form part of the present specification and are included to further demonstrate certain aspects of the present invention. The invention may be better understood by reference to these drawings in combination with the detailed description of specific embodiments presented herein.

FIG. 1A and FIG. 1B show the preservation of the ERG function at 5 days (FIG. 1A) and 1 month (FIG. 1B) in rats dosed systemically with safinamide and exposed to a severe photo-oxidative insult. Dosing of 15-60 mg/kg safinamide provided significant and complete retinal function protection.

FIG. 2 shows the prevention of retinal lesions by treatment with safinamide. Rats dosed with 60 mg/kg of safinamide were devoid of significant retinal lesions.

### DESCRIPTION OF THE INVENTION

### Chemical Part

Object of the present invention are compounds of the general formula I

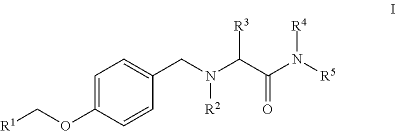

wherein:
R¹ is C5-C7 cycloalkyl; phenyl (unsubstituted) or phenyl substituted independently with one or more halogens or CF3
R² is H, C1-C3 alkyl
R³ is H, C1-C3 alkyl (unsubstituted) or C1-C3 alkyl substituted with OR⁶
R⁴, R⁵ are, independently H, C1-C3 alkyl
R⁶ is H, C1-C2 alkyl

Preferred compounds of formula I are compounds wherein:
R¹ is C5-C7 cycloalkyl; phenyl (unsubstituted) or phenyl substituted independently with one or two F, Cl or CF3
R² is H, C1-C2 alkyl
R³ is H, C1-C2 alkyl (unsubstituted) or C1-C2 alkyl substituted with OR⁶
R⁴, R⁵ are independently H, C1-C2 alkyl
R⁶ is H, C1-C2 alkyl

More preferred compounds of formula I are compounds wherein:
R¹ is phenyl (unsubstituted) or phenyl substituted independently with one or two F, Cl
R² is H, CH₃
R³ is H, C1-C2 alkyl (unsubstituted) or C1-C2 alkyl substituted with OR⁶
R⁴, R⁵ are independently H, CH₃
R⁶ is H, CH₃

Most preferred compounds of formula I are the (S) and (R) compounds wherein:
R¹ is 3-fluorophenyl
R² is H
R³ is CH₃
R⁴, R⁵ are H
   and particularly the (S)-isomer (safinamide).

The compounds of formula I are known compounds and are prepared according to the methods described in U.S. Pat. No. 5,263,957.

### Biological Part

Retinal diseases are often disruptive to the tissue and can result in a loss of visual function for millions of patients. For example, retinal tissues can be damaged by environmental factors, such as light exposure, which is known to contribute to the to progression of retinal degenerative disorders such as AMD (Young 1988; Taylor et al. 1992; Cruickshank et al. 1993). To date, no effective treatment exists for neurodegenerative disorders of the retina. Early stages of macular degeneration are typically treated by combinations of antioxidants or anti-inflammatory agents whose efficacy has not been demonstrated in the clinic. Advanced stages of macular degeneration that lead to severe vision loss are treated either by surgical removal of membranes from the subretinal space, laser photocoagulation, photodynamic therapy, and most recently with VEGF blockers in patients with exudative AMD. No approved treatments are available for the advanced form of dry AMD known as Geographic Atrophy. Laser treatment is also used in the treatment of diabetic retinopathy. It is important to note that both laser photocoagulation of the retina and surgical excision of subretinal membranes or intravitreal membranes results in the destruction of viable retinal neurons. Prevention or reduction of outer retina damage by MAO inhibitors is a unique and novel therapeutic approach to the management of age-related maculopathy and/or macular degeneration and other retinopathies.

In light damage paradigms used by the present inventors, antioxidants were either ineffective (α-tocopherol) or marginally effective at high doses (ascorbate, vitamin E analogs). Similarly, some calcium antagonists (flunarizine, nicardipine) were moderately effective while others (nifedipine, nimodipine, verapamil) had no effect in preventing light-induced functional or morphological changes. Unexpectedly, it has been discovered that compounds of this invention are 50 to 100-fold more potent than antioxidants in this light damage paradigm and therefore are useful for treating disorders of the outer retina.

Monoamine oxidase (MAO) is an integral protein of the outer mitochondrial membrane and plays a major role in the inactivation of amines in the central nervous system (CNS) and peripheral nervous system (PNS). In the human CNS, the MAO-A isoenzyme is responsible for deamination of serotonin and noradrenaline, while the MAO-B isoenzyme is responsible for deamination of dopamine. After initial enthusiasm, the use of MAO-A and nonselective MAO inhibitors has been limited by the wide range of MAO induced-drug and MAO induced-food interactions that are possible, particularly with sympathomimetic medications or tyramine-containing foods, resulting in hypertensive reactions. Inhibitors of the MAO-B isoenzyme have demonstrated neuroprotective and neurorescuing properties in a number of models, including: monkey and mouse MPTP model, mouse head injury model; facial nerve axotomy in rats; and acute drug-induced dopaminergic motor dysfunction in rodents. Long acting MAO-B inhibitors (deprenyl, selegiline) have also been associated with insomnia, nausea, benign cardiac arrhythmias, dizziness and headache.

The invention contemplates the use of the MAO inhibitor of general formula I or any pharmaceutically acceptable derivative, including pharmaceutically acceptable salts, for treating disorders of the outer retina. The phrase “pharmaceutically acceptable” means the compounds can be safely used for the treatment of diseases of the outer retina. As used herein, the outer retina includes the RPE, photoreceptors, Muller cells (to the extent that their processes extend into the outer retina), and the outer plexiform layer. The compounds are formulated for systemic or local ocular delivery.

While it is contemplated that any short acting inhibitors of MAO A/B and B will be useful in the methods of the present invention, preferred MAO inhibitors are potent, short acting inhibitors of the MAO-B receptor, such as those compounds described specifically herein. Preferred compounds include 2-{[4-(3-Chloro-benzyloxy)-benzyl]-methyl-amino}-acetamide, (S)-2-[4-(2-Fluoro-benzyloxy)-benzylamino]-propionamide, (S)-2-[4-(4-Fluoro-benzyloxy)-benzylamino]-propionamide, (S)-2-[4-(3-Chloro-benzyloxy)-benzylamino]-propionamide, (R)-2-[4-(3-Chloro-benzyloxy)-benzylamino]-3-hydroxy-propionamide, (S)-2-(4-Cyclohexylmethoxy-benzylamino)-propionamide, (S)-2-[4-(3-Fluoro-benzyloxy)-benzylamino]-3-hydroxy-propionamide, (S)-2-[4-(3-Chloro-benzyloxy)-benzylamino]-3-hydroxy-propionamide, (S)-2-{[4-(3-Chloro-benzyloxy)-benzyl]-methyl-amino}-propionamide, (S)-2-[4-(3-Fluoro-benzyloxy)-benzylamino]-propionamide (safinamide), (S)-2-[4-(3-Fluoro-benzyloxy)-benzylamino]-propionamide (safinamide) or any pharmaceutically acceptable derivative or analog or salt of these compounds. The most preferred compound for use in the methods described herein is safinamide or any pharmaceutically acceptable derivative, analog or salt thereof.

Disorders of the outer retina encompass acute and chronic environmentally induced (trauma, ischemia, photo-oxidative stress) degenerative conditions of the photoreceptors and RPE cells in normal or genetically predisposed individuals. Such disorders include, but are not limited to, age-related macular degeneration (AMD); retinitis pigmentosa (RP) and other forms of heredodegenerative retinal disease; retinal detachment; tears; macular pucker; ischemia affecting the outer retina; diabetic retinopathy; damage associated with laser therapy (grid, focal and panretinal) including photodynamic therapy (PDT), thermal or cryotherapy; trauma; surgical (retinal translocation, subretinal surgery or vitrectomy) or light induced iatrogenic retinopathy; and preservation of retinal transplants.

The compounds of this invention, which are potent and selective inhibitors of MAO-B (IC50 in the submicromolar-nanomolar range), in vitro and in vivo, have generally no relevant effect on MAO-A. After oral administration in mice, the compounds behave as potent, short-acting MAO-B inhibitors with full recovery of activity 8-16 hours after administration of a single dose of substance.

The MAO inhibiting activity of compounds useful for the methods of the present invention may be determined using a variety of methods known to the skilled artisan. Method 1 and Method 2, described below are examples of useful assays for determining MAO B inhibiting activity.

### Method 1

### In Vitro MAO-A and MAO-B Enzyme Activities Assay

### —Membrane Preparations (Crude Mitochondrial Fraction)

Male Wistar rats (Harlan, Italy—175-200 g) were sacrificed under light anaesthesia and brains were rapidly removed and homogenized in 8 volumes of ice-cold 0.32 M sucrose buffer containing 0.1 M EDTA, pH 7.4. The crude homogenate was centrifuged at 2220 rpm for 10 minutes at +4° C. and the supernatant recovered. The pellet was homogenized and centrifuged again. The two supernatants were pooled and centrifuged at 9250 rpm for 10 minutes. The pellet was resuspended in fresh buffer and centrifuged at 11250 rpm for 10 minutes at +4° C. The resulting pellet was stored at −80° C.

### —In Vitro Enzyme Activities Assay

The enzyme activities were assessed with a radioenzymatic assay using the substrates ¹⁴C-serotonin (5-HT) and ¹⁴C-phenylethylamine (PEA) for MAO-A and MAO-B, respectively.

The mitochondrial pellet (500 μg protein) was resuspended in 0.1 M phosphate buffer (pH 7.4). 500 μl of the suspension were added to a 50 μl solution of the test compound or buffer, and incubated for 30 min at 37° C. (preincubation) then the substrate (50 μl) was added. The incubation was carried out for 30 minutes at 37° C. (¹⁴C-5-HT, 5 μM) or for 10 minutes at 37° C. (¹⁴C-PEA, 0.5 μM).

The reaction was stopped by adding 0.2 ml of 37% HCl or perchloric acid. After centrifugation, the deaminated metabolites were extracted with 3 ml of diethyl ether (5-HT) or toluene (PEA) and the radioactive organic phase was measured by liquid scintillation spectrometry at 90% efficiency. The amount of neutral and/or acidic metabolites formed as a result of MAO activity was obtained by measuring the radioactivity of the eluate.

The activity of MAO in the sample, corresponding to a percentage of radioactivity compared with the control activity in the absence of the inhibitor, was expressed as nmoles of substrate transformed/mg protein/min.

The drug inhibition curves were obtained from at least eight different concentration points, each in duplicate (10⁻¹⁰ to 10⁻⁵ M). The IC50 values (the drug concentration inhibiting 50% of the enzyme activity) were calculated with confidence intervals determined using non linear regression analysis (best fitting aided-computer program).

The procedure described in Method 1 was used to generate the data shown in Table 1.

### Method 2

### Ex Vivo MAO-B Inhibition

Test compounds were administered orally to male C57BL mice (Harlan, Italy, 25-27 g) at the single dose of 20 mg/Kg. At various time intervals (1, 2, 4, 8 and 24 h), animals were sacrificed, brains removed, cortices dissected out and stored at −80° C. Crude homogenates (0.5%) were prepared in 0.1 M phosphate buffer (pH 7.4) and were freshly used. MAO-A and MAO-B activity were assessed as described above.

TABLE 1
In vitro MAO-A and MAO-B inhibition of some compounds
of the invention in rat brain mitochondria

| | |
|---|---|
| MAO A | MAO B |

| | |
|---|---|
| COMPOUND | IC₅₀, μM |

| | | |
|---|---|---|
| 2-{[4-(3-Chloro-benzyloxy)-benzyl]- | 50 | 0.04 |
| methyl-amino}-acetamide | | |
| (S)-2-[4-(2-Fluoro-benzyloxy)- | 228 | 0.18 |
| benzylamino]-propionamide | | |
| (S)-2-[4-(4-Fluoro-benzyloxy)- | 93 | 0.06 |
| benzylamino]-propionamide | | |
| (S)-2-[4-(3-Chloro-benzyloxy)- | 114 | 0.03 |
| benzylamino]-propionamide | | |
| (R)-2-[4-(3-Chloro-benzyloxy)- | >100 | 0.11 |
| benzylamino]-3-hydroxy-propionamide | | |
| (S)-2-(4-Cyclohexylmethoxy- | 301 | 0.03 |
| benzylamino)-propionamide | | |
| (S)-2-[4-(3-Fluoro-benzyloxy)- | 100 | 0.14 |
| benzylamino]-3-hydroxy-propionamide | | |
| (S)-2-[4-(3-Chloro-benzyloxy)- | 84 | 0.04 |
| benzylamino]-3-hydroxy-propionamide | | |
| (S)-2-{[4-(3-Chloro-benzyloxy)-benzyl]- | 82 | 0.06 |
| methyl-amino}-propionamide | | |
| (S)-2-[4-(3-Fluoro-benzyloxy)- | 584 | 0.09 |
| benzylamino]-propionamide (safinamide) | | |

### Method 3

### Neuroprotective Activity of MAO Inhibitors in the Rat Photo-Oxidative Induced Retinopathy Model

Photic retinopathy results from excessive excitation of the RPE and neuroretina by absorption of visible or near ultraviolet radiation. Lesion severity is dependent upon wavelength, irradiance, exposure duration, species, ocular pigmentation, and age. Damage may result from peroxidation of cellular membranes, inactivation of mitochondrial enzymes such as cytochrome oxidase, and/or increased intracellular calcium. Cellular damage resulting from photo-oxidative stress leads to cell death by apoptosis, (Shahinfar, et al., 1991, Current Eye Research, Vol. 10:47-59; Abler, et al., 1994, Investigative Ophthalmology & Visual Science, Vol. 35(Suppl):1517). Oxidative stress induced apoptosis has been implicated as a cause of many ocular pathologies, including, iatrogenic retinopathy, macular degeneration, RP and other forms of heredodegenerative disease, ischemic retinopathy, retinal tears, retinal detachment, glaucoma and retinal neovascularization (Chang, et al., 1995, Archives of Ophthalmology, Vol. 113:880-886; Portera-Cailliau, et al., 1994, Proceedings of National Academy of Science (U.S.A.), Vol. 91:974-978; Buchi, E. R., 1992, Experimental Eye Research, Vol. 55:605-613; Quigley, et al., 1995, Investigative Ophthalmology & Visual Science, Vol. 36:774-786). Photic induced retinal damage has been observed in mice (Zigman, et al., 1975, Investigative Ophthalmology & Visual Science, Vol. 14:710-713), rats (Noell, et al., 1966, Investigative Ophthalmology and Visual Science, Vol. 5:450-473; Kuwabara, et al., 1968, Archives of Ophthalmology, Vol. 79:69-78; LaVail, M. M., 1976, Investigative Ophthalmology & Visual Science, Vol. 15:64-70), rabbit (Lawwill, T., 1973, Investigative Ophthalmology & Visual Science, Vol. 12:45-51), and squirrel (Collier, et al., 1989; In LaVail et al., Inherited and Environmentally Induced Retinal Degenerations. Alan R. Liss, Inc., New York; Collier, et al., 1989, Investigative Ophthalmology & Visual Science, Vol. 30:631-637), non-human primates (Tso, M. O. M., 1973, Investigative Ophthalmology & Visual Science, Vol. 12:17-34; Ham, et al., 1980, Vision Research, Vol. 20:1105-1111; Sperling, et al., 1980, Vision Research, Vol. 20:1117-1125; Sykes, et al., 1981, Investigative Ophthalmology & Visual Science, Vol. 20:425-434; Lawwill, T., 1982, Transactions of the American Ophthalmology Society, Vol. 80:517-577), and man (Marshall, et al., 1975, British Journal of Ophthalmology, Vol. 59:610-630; Green, et al., 1991, American Journal of Ophthalmology, Vol. 112:520-27). In man, chronic exposure to environmental radiation has also been implicated as a risk factor for ARMD (Young, R. W., 1988, Survey of Ophthalmology, Vol. 32:252-269; Taylor, et al., 1992, Archives of Ophthalmology, Vol. 110:99-104; Cruickshank, et al., 1993, Archives of Ophthalmology, Vol. 111:514-518)

Prevention of Photo-oxidative injury with Safinamide: The efficacy of Safinamide, a short acting MAO-B inhibitor, to protect retinal cells against the induction of photochemical lesions by blue-light exposure was assessed by measuring light-induced changes in retinal functioning (electroretinogram (ERG)) and evaluating retinal morphology changes. Significant dose-dependent protection of retinal function was measured in light exposed rats after a 5-day recovery period in rats dosed with Safinamide (5-60 mg/kg). ERGs were not significantly different from normal after a 1-month recovery period in Safinamide dosed rats (15 to 60 mg/kg).

Subjects. Male Sprague Dawley rats were randomly assigned to drug or vehicle experimental groups. Rats receiving vehicle (N=15) or drug treatment (Safinamide: 5 mg/kg, N=10; 15 mg/kg, N=10; 30 mg/kg, N=10; and 60 mg/kg, N=9) were pre-dosed (IP) at 48, 24 and 0 hours prior to a 6-hour light exposure (spectrally filtered blue light (˜220 fc)) and 24 and 48 hours after light exposure. Control rats were housed in their home cage under normal cyclic light exposure. Control rats were not dosed with either vehicle or drug.

Retinal Function Assessment. The ERG is a non-invasive clinical measurement of the electrical response of the eye to a flash of light. The a-wave and b-wave are two components of the ERG that are diagnostic of retinal function. The a-wave reflects outer retina function and is generated by interactions between photoreceptor and RPE while the b-wave reflects inner retina function, particularly on-bipolar cells. Although the inner retina is not significantly damaged by this light exposure, the b-wave is depressed due to the lack of photoreceptor input. Changes in the a-wave amplitude or latency are diagnostic of outer retina pathology. The ERG was recorded after a five day recovery period from dark-adapted anesthetized rats (ketamine-HCl, 75 mg/Kg; xylazine, 6 mg/Kg). The eye's electrical response to a flash of light was elicited by viewing a ganzfeld. ERGs to a series of light flashes increasing in intensity were digitized to analyze temporal characteristics of the waveform and response voltage-log intensity relationship.

Light Microscopic Assessment of Retinal Lesions. Ocular tissues were fixed in a mixture of paraformaldehyde and glutaraldehyde, dehydrated in an ascending ethanol series, embedded in JB-4 plastic resin, and 1 to 1.5-micron thick sections were analyzed using a quantitative computer image analysis system attached to the microscope. Retinal Pigment Epithelium (RPE), Outer Nuclear Layer (ONL) and Inner to Nuclear Layer (INL) thickness as well as the length of inner segments (IS), where mitochondria are located, and outer segments (OS), which contain the light sensitive photopigment, were measured to assess outer retina protection. As the INL is not significantly affected by light exposure, this layer served as an additional control measurement.

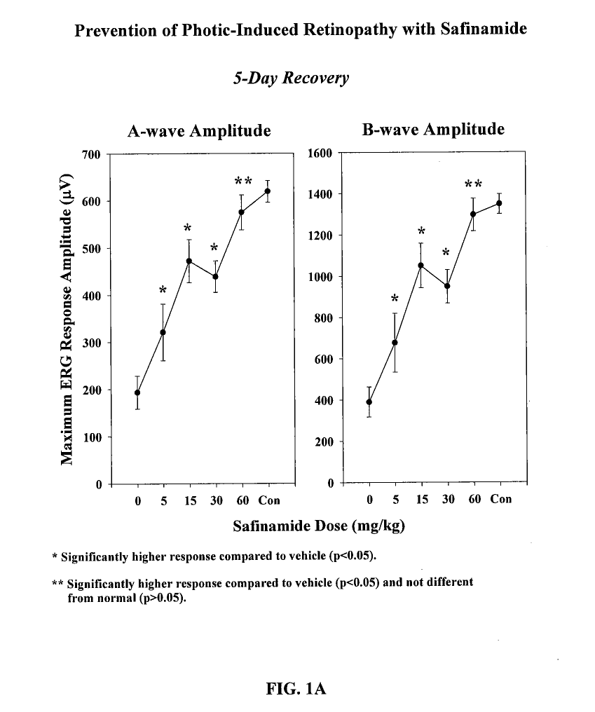

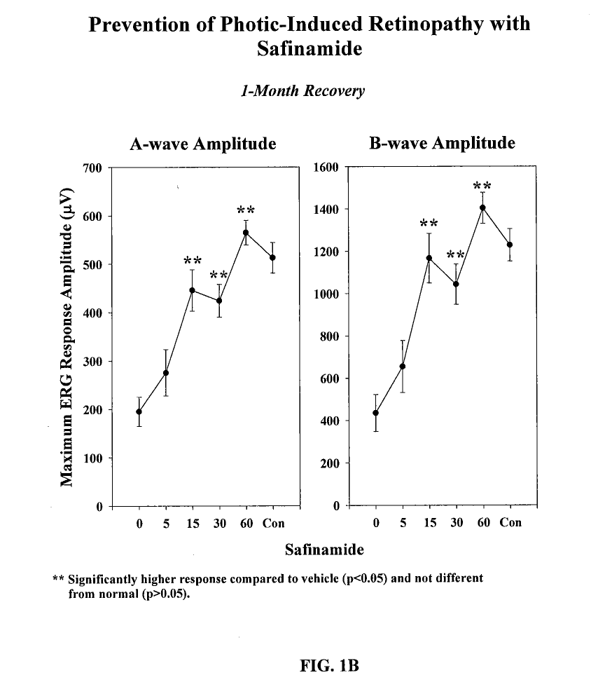

Results. Blue-light exposure to vehicle-dosed rats resulted in a significant reduction in retinal function (ANOVA, p<0.001), as measured by the ERG, when measured 5 days after light exposure (FIG. 1A). After blue-light exposure, maximum a-wave response amplitudes were reduced 69% and maximum b-wave response amplitudes were reduced 71% from vehicle-dosed rats. Dosing with Safinamide resulted in dose-dependent protection of retinal function (FIG. 1A). At all doses evaluated (5-60 mg/kg), significant ERG protection was measured compared to vehicle-dosed rats. Maximum ERG a-wave responses from rats dosed with Safinamide (5 mg/kg) were 52% of normal and responses recorded from rats dosed with 15 or 30 mg/kg were greater than 70% of normal. Retinal responses from Safinamide (60 mg/kg) dosed rats were not significantly diminished compared to control rats that were maintained under normal, dim, visible, cyclic light.

FIG. 1A shows ERG response amplitudes measured 5 days after a 6-hour blue-light exposure. Dosing with Safinamide (5-60 mg/kg) provided significant retinal function protection.

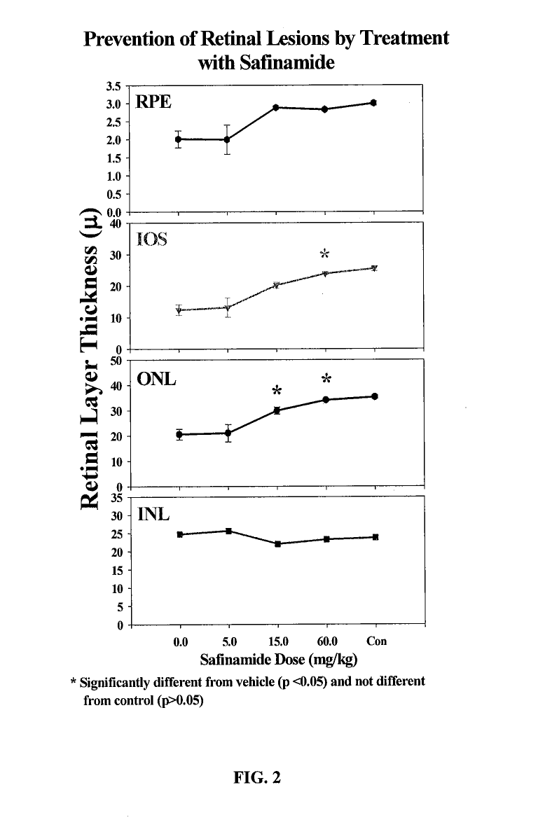

After an additional 3-week recovery period, evaluation of the flash-induced retinal response (FIG. 1B) demonstrated no significant recovery of ERG responses from vehicle-dosed rats. Evaluation of the ERG response in Safinamide (15 to 60 mg/kg) dosed rats demonstrated normal ERG a- and b-wave function. Light microscopic evaluation of retinas from vehicle-dosed rats demonstrated significant (ANOVA, p<0.001) thinning of the RPE as well as loss of photoreceptor cells and shortening of their inner+outer segment length (FIG. 2). Dose-dependent reduction in retinal lesions were measured in rats dosed with Safinamide. Retinas obtained from rats dosed with Safinamide (15 and 60 mg/kg) demonstrated a thicker RPE compared to vehicle-dosed rats. The ONL was significantly thicker in rats dosed with Safinamide (15 and 60 mg/kg) and photoreceptor segment length was significantly longer (60 mg/kg) compared to vehicle-dosed rats and not significantly different from normal controls. Retinas from rats dosed with Safinamide (60 mg/kg) were devoid of any significant retinal lesions.

In general, for degenerative diseases, the compounds of this invention are administered orally with daily dosage of these compounds ranging between about 0.001 and about 500 milligrams. The preferred total daily dose ranges between about 1 and about 100 milligrams. Non-oral administration, such as, intravitreal, topical ocular, transdermal patch, subdermal, parenteral, intraocular, subconjunctival, or retrobulbar or subtenon's injection, trans scleral (including iontophoresis), posterior juxtascleral delivery, or slow release biodegradable polymers or liposomes may require an adjustment of the total daily dose necessary to provide a therapeutically effective amount of the compound. The Compounds can also be delivered in ocular irrigating solutions. Concentrations should range from about 0.001 μM to about 100 μM, preferably about 0.01 μM to about 5

The compounds can be incorporated into various types of ophthalmic formulations for delivery to the eye (e.g., topically, intracamerally, juxtasclerally, or via an implant). They may be combined with ophthalmologically acceptable preservatives, surfactants, viscosity enhancers, gelling agents, penetration enhancers, buffers, sodium chloride, and water to form aqueous, sterile ophthalmic suspensions or solutions or preformed gels or gels formed in situ. Ophthalmic solution formulations may be prepared by dissolving the compound in a physiologically acceptable isotonic aqueous buffer. Further, the ophthalmic solution may include an ophthalmologically acceptable surfactant to assist in dissolving the compound. The ophthalmic solutions may contain a viscosity enhancer, such as, hydroxymethylcellulose, hydroxyethylcellulose, hydroxypropylmethylcellulose, methylcellulose, polyvinyl-pyrrolidone, or the like, to improve the retention of the formulation in the conjunctival sac. In order to prepare sterile ophthalmic ointment formulations, the active ingredient is combined with a preservative in an appropriate vehicle, such as, mineral oil, liquid lanolin, or white petrolatum. Sterile ophthalmic gel formulations may be prepared by suspending the active ingredient in a hydrophilic base prepared from the combination of, for example, carbopol-940, or the like, according to the published formulations for analogous ophthalmic preparations; preservatives and tonicity agents can be incorporated.

If dosed topically, the compounds are preferably formulated as topical ophthalmic suspensions or solutions, with a pH of about 4 to 8. The Compounds will normally be contained in these formulations in an amount 0.001% to 5% by weight, but preferably in an amount of 0.01% to 2% by weight. Thus, for topical presentation, 1 to 2 drops of these formulations would be delivered to the surface of the eye 1 to 4 times per day according to the discretion of a skilled clinician.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

The following topical ophthalmic formulations are useful according to the present invention administered 1-4 times per day according to the discretion of a skilled clinician.

### Example 1

| Ingredients | Amount (wt %) |
|---|---|
| Safinamide | 0.01-2% |
| Hydroxypropyl methylcellulose | 0.5% |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting pH to 7.3-7.4 |
| Purified water | q.s. to 100% |

### Example 2

| Ingredients | Amount (wt %) |
|---|---|
| Safinamide | 0.01-2% |
| Methyl cellulose | 4.0% |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting pH to 7.3-7.4 |
| Purified water | q.s. to 100% |

### Example 3

| Ingredients | Amount (wt %) |
|---|---|
| Compound | 0.01-2% |
| Guar gum | 0.4-6.0% |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting pH to 7.3-7.4 |
| Purified water | q.s. to 100% |

### Example 4

| Ingredients | Amount (wt %) |
|---|---|
| Compound | 0.01-2% |
| White petrolatum and mineral oil and lanolin | Ointment consistency |
| Dibasic sodium phosphate (anhydrous) | 0.2% |
| Sodium chloride | 0.5% |
| Disodium EDTA (Edetate disodium) | 0.01% |
| Polysorbate 80 | 0.05% |
| Benzalkonium chloride | 0.01% |
| Sodium hydroxide/Hydrochloric acid | For adjusting pH to 7.3-7.4 |

### Example 5

10 mM IV Solution w/v %

| | |
|---|---|
| Safinamide | 0.384% (about 4%) |
| L-Tartaric acid | 2.31% |
| Sodium hydroxide | pH 3.8 |
| Hydrochloric acid | pH 3.8 |
| Purified water | q.s. 100% |

### Example 6

5 mg Capsules

| | mg/capsule |
|---|---|
| Ingredient | (Total Wt. 22a mg) |
| Safinamide | 5 |
| Lactose, anhydrous | 55.7 |
| Strach, Sodium carboxy-methyl | 8 |
| Cellulose, microcrystalline | 30 |
| Colloidal silicon dioxide | .5 |
| Magnesium sterage | .8 |

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and structurally related may be substituted for the agents described herein to achieve similar results. All such substitutions and modifications apparent to those skilled in the art are deemed to be within the spirit, scope and concept of the invention as defined by the appended claims.

The references cited herein, to the extent that they provide exemplary procedural or other details supplementary to those set forth herein, are specifically incorporated herein by reference.

We claim:

1. A method for treating disorders of the outer retina which comprises administering a composition comprising a therapeutically effective amount of a monoamine oxidase inhibitor, wherein said monoamine oxidase inhibitor is a compound of formula I or a pharmaceutically acceptable derivative or analogue thereof 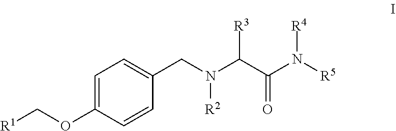
wherein:
R¹ is C5-C7 cycloalkyl; phenyl (unsubstituted) or phenyl substituted independently with one or more halogens or CF3
R² is H, C1-C3 alkyl
R³ is H, C1-C3 alkyl (unsubstituted) or C1-C3 alkyl substituted with OR⁶
R⁴, R⁵ are, independently H, C1-C3 alkyl
R⁶ is H, C1-C2 alkyl.

2. The method claim 1 wherein:
R¹ is C5-C7 cycloalkyl; phenyl (unsubstituted) or phenyl substituted independently with one or two F, Cl or CF3
R² is H, C1-C2 alkyl
R³ is H, C1-C2 alkyl (unsubstituted) or C1-C2 alkyl substituted with OR⁶
R⁴, R⁵ are independently H, C1-C2 alkyl
R⁶ is H, C1-C2 alkyl.

3. The method of claim 1 wherein:
R¹ is phenyl (unsubstituted) or phenyl substituted independently with one or two F, Cl
R² is H, CH₃
R³ is H, C1-C2 alkyl (unsubstituted) or C1-C2 alkyl substituted with OR⁶
R⁴, R⁵ are independently H, CH₃
R⁶ is H, CH₃.

4. The method of claim 3 wherein:
R¹ is 3-Fluorophenyl
R² is H
R³ is CH₃
R⁴, R⁵ are H.

5. The method of claim 4, wherein the compound is safinamide.

6. The method of claim 1 wherein the disorder is selected from the group consisting of: AMD; RP and other forms of heredodegenerative retinal disease; retinal to detachment and tears; macular pucker; ischemia affecting the outer retina; diabetic retinopathy; damage associated with laser therapy (grid, focal, and panretinal) including photodynamic therapy (PDT); trauma; surgical (retinal translocation, subretinal surgery, or vitrectomy) or light-induced iatrogenic retinopathy; and preservation of retinal transplants.

7. The method of claim 6, wherein the disorder is selected from the group consisting of AMD, RP, and diabetic retinopathy.

8. The method of claim 7 wherein the disorder is AMD.

9. The method of claim 1, wherein the amount of monoamine oxidase inhibitor in the composition is from about 0.01 to about 2%.

10. The method of claim 1, wherein the administration is via a method selected from the group consisting of topical ocular administration, intravitreal injection, oral administration, retrobulbar administration, subconjunctival administration, subtenon administration, transdermal administration, intravenous administration, intraperitoneal administration, subcutaneous administration, administration via slow release biodegradable polymers, liposomes, and via mini-pumps.

11. The method of claim 10, wherein the administration is via local delivery.

12. A method of treating or preventing retinal degeneration, said method comprising administering to a patient a composition comprising a therapeutically effective amount of a monoamine oxidase inhibitor, wherein said monoamine oxidase inhibitor is a compound of formula I, or a pharmaceutically acceptable derivative or analog thereof 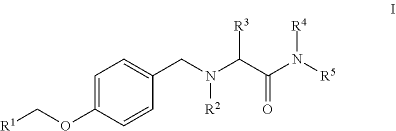
wherein:
R¹ is C5-C7 cycloalkyl; phenyl (unsubstituted) or phenyl substituted independently with one or more halogens or CF3
R² is H, C1-C3 alkyl
R³ is H, C1-C3 alkyl (unsubstituted) or C1-C3 alkyl substituted with OR⁶
R⁴, R⁵ are, independently H, C1-C3 alkyl
R⁶ is H, C1-C2 alkyl.

13. The method of claim 12, wherein the monoamine oxidase inhibitor is safinamide.

14. The method of claim 12, wherein the disorder is selected from the group consisting of: AMD; RP and other forms of heredodegenerative retinal disease; retinal detachment and tears; macular pucker; ischemia affecting the outer retina; diabetic retinopathy; damage associated with laser therapy (grid, focal, and panretinal) including photodynamic therapy (PDT); trauma; surgical (retinal translocation, subretinal surgery, or vitrectomy) or light-induced iatrogenic retinopathy; and preservation of retinal transplants.

15. The method of claim 14, wherein the disorder is selected from the group consisting of AMD, RP, and diabetic retinopathy.

16. The method of claim 15, wherein the disorder is AMD.

17. The method of claim 12, wherein the amount of monoamine oxidase inhibitor in the composition is from about 0.01% to about 2%.

18. The method of claim 12, wherein the administration is via a method selected from the group consisting of topical ocular administration, intravitreal injection, oral administration, retrobulbar administration, subconjunctival administration, subtenon administration, transdermal administration, intravenous administration, intraperitoneal administration, subcutaneous administration, administration via slow release biodegradable polymers, liposomes, and via mini-pumps.

19. The method of claim 18, wherein the administration is via local delivery.
